(19) United States
(12) Patent Application Publication
(10) Pub. No.: US 2026/0357266 A1
(43) Pub. Date: Nov. 12, 2026

(54) COMPOSITION FOR KILLING A THERAPY-INDUCED SENESCENCE (TIS) CELLS AND USES THEREOF

(71) Applicant: AJOU UNIVERSITY INDUSTRY-ACADEMIC COOPERATION FOUNDATION, Suwon-si, Gyeonggi-do (KR)

(72) Inventors: Yong Won CHOI, Yongin-si (KR); Tae Jun PARK, Seongnam-si (KR); Dong Hyun LEE, Suwon-si (KR)

(73) Assignee: AJOU UNIVERSITY INDUSTRY-ACADEMIC COOPERATION FOUNDATION, Suwon-si, Gyeonggi-do (KR)

(21) Appl. No.: 19/122,901

(22) PCT Filed: Oct. 18, 2023
(86) PCT No.: PCT/KR2023/016121

§ 371 Date: Apr. 21, 2025

(30) Foreign Application Priority Data
Oct. 20, 2022 (KR) 10-2022-0135645

(51) Int. Cl. C12N 15/113; A61P 3/00

(52) U.S. Cl. CPC C12N 15/1137; A61P 3/00; C12N 2310/14

(57) ABSTRACT

A composition for killing therapy-induced senescent cells, containing a Pim-1 kinase inhibitor, and uses thereof for preventing or treating senescence-related disease or cancer are disclosed. The composition for killing senescent cells containing a Pim-1 kinase inhibitor exhibits a selective killing effect on senescent cells, particularly senescent cells with increased expression of Pim-1 kinase. The composition for killing senescent cells may be effectively used to prevent or treat senescence-related diseases through its selective killing effect on senescent cells. In particular, the composition may exhibit an excellent effect on the prevention or treatment of tumors by selectively killing therapy-induced senescent cells by administering the Pim-1 kinase inhibitor subsequent to anticancer therapy based on the induction of senescence of tumor cells.

### TECHNICAL FIELD

The present invention relates to a composition for killing therapy-induced senescent cells and the use thereof, and more specifically, to a composition for killing therapy-induced senescent cells, comprising a Pim-1 kinase inhibitor, and the use thereof for preventing or treating senescence-related disease or cancer.

### BACKGROUND ART

Senescence refers to a state of stable cell cycle arrest. Senescence is characterized by the senescence-associated secretory phenotype (SASP), such as the secretion of proinflammatory cytokines, growth factors, and matrix metalloproteinases (MMPs) (Kopp, H. G. et al., Histol. Histopathol. 22, 971-976 (2007)). From an oncological perspective, senescence is known as a powerful protective mechanism against tumorigenesis. Oncogene-induced senescence (OIS) induced by oncogenes such as HRASV12 inhibits the growth and proliferation of cells at risk for malignant transformation, thereby inhibiting the development of malignant tumors. Loss of senescence functions due to mutations in these senescence-related oncogenes can cause tumors (Serrena, M, et al., Cell 88, 593-602 (1997).

Senescence of tumor cells is used as a major mechanism of various anticancer therapies for the treatment of tumors, such as chemotherapy and radiotherapy (Wang, B., Kohli, J. & Demaria, M. Trends Cancer 6, 838-857 (2020)). Various chemotherapy and radiotherapy regimens reported in academia and clinical practice induce cellular senescence as a primary response to the therapy regimens. For example, therapies such as hyperactive oncogenic signaling, radiotherapy, traditional chemotherapy, CDC7 inhibitors, and telomerase inhibitors induce senescence through DNA damage and p53/p21 signaling. This p53/p21 signaling-mediated senescence can be induced by drugs such as PTEN inhibitors such as VO-OHpi, MDM2-p53 interaction disruptors such as nutlin 3, RG7112 or UBX0101, and histone deacetylase inhibitors such as vorinostat. In addition, DNA methyltransferase inhibitors such as decitabine, CDK inhibitors, aurora kinase inhibitors (AURKs), and PLK1 inhibitors induce senescence by blocking G2/M progression of the cell cycle.

It is known that therapy-induced senescent (TIS) cancer cells inhibit the growth and proliferation of tumor cells by affecting the tumor microenvironment through various mechanisms, such as arresting adjacent cancer cells through SASP, improving vascular structures for drug delivery, and recruiting immune cells (Faget, D. V. et al., Nat. Rev. Cancer 19, 439-453 (2019); Krizhanovsky, V. et al. Cell 134, 657-667 (2008); Ritschka, B. et al., Genes Dev. 31, 172-183 (2017); Chang, B. D. et al. Oncogene 18, 4808-4818 (1999)).

Meanwhile, it has been reported that, although therapy-induced senescence (TIS) and TIS-induced SASP initially exhibit antitumor effects, they may exhibit pro-tumorigenic characteristics in the long term, such as induction of inflammation, creation of an immunosuppressive microenvironment, promotion of angiogenesis, and metastasis of adjacent cancer cells (Coppe, J. P. et al. PLoS Biol. 6, 2853-2868 (2008); Yang, L., Discov. 3, 17049 (2017); Watanabe, S., et al., Cancer Sci. 108, 563-569 (2017)). Specifically, MMPs, known as SASP factors, can promote angiogenesis, and cancer cell growth and invasion (Open Biol. 11, 200360 (2021)), and IL-6 and IL-8 can form a chronic inflammatory microenvironment, which can induce tumorigenesis and various side effects.

Accordingly, a therapeutic method that selectively kills therapy-induced senescent cells, thereby preventing long-term side effects of senescent cells, has been reported. This method, also called “one-two punch” therapy, is a new therapeutic method that induces the senescence of tumor cells through recent chemotherapy and radiotherapy to induce a primary therapeutic effect through an early anti-tumor effect, and subsequently selectively kills senescent cells (senolytic therapy), thereby preventing tumor recurrence and long-term side effects (Nature Reviews Cancer volume 22, pages 340-355 (2022)). Since the “one-two punch” therapy is not based on co-administration of drugs but on sequential treatment using each drug, it has advantages in that it can avoid combination toxicity caused by a combination of drugs, and thus enables the use of various senolytic drugs that have been previously reported.

Currently, various studies are being conducted to use previously reported senolytic drugs in “one-two punch” therapy for the treatment of tumors. Specifically, it has been reported that navitoclax (ABT-263) and selective inhibitors of BCL-2, BCL-XL, and BCL-W are able to effectively eliminate a wide range of senescent cell types, including senescent cancer cells (Aging Cell 15, 428-435 (2016); Nat. Med. 22, 78-83 (2016); U.S. Ser. No. 11/331,328B). Combination therapy of rituximab, a tumor senescence inducer, and navitoclax showed high efficacy and excellent tolerability in a phase 2 study (Leuk. Lymphoma 56, 2826-2833 (2015)).

Despite the remarkable effect of “one-two punch” therapy, there is a significant lack of senolytic drugs reported to date that can be used to treat tumors (Nature Reviews Cancer volume 22, pages 340-355 (2022)). In particular, currently reported navitoclax, ABT737, NAav-Gal, etc. are all drugs that kill senescent cells through BCL-2/XL/W-based pharmacological mechanisms, and thus they may indiscriminately act on various senescent cells other than therapy-induced senescent cells, causing unexpected side effects. Accordingly, there is an increasing need for the development of drugs that kills senescent cells through various mechanisms, the discovery of biomarkers specific to therapy-induced senescent tumor cells, and the development of drugs that specifically kill therapy-induced senescent cells based thereon.

Under this background art, the present inventors have made extensive efforts to discover a novel biomarker of therapy-induced senescent tumor cells and to develop a novel senolytic drug that may be used in “one-two punch” therapy, and as a result, have found that the expression of Pim-1 kinase is commonly increased in therapy-induced senescent tumor cells induced by various anti-tumor therapies, and thus it may be used as a novel biomarker of therapy-induced senescent tumor cells, and furthermore, that treatment with a Pim-1 kinase inhibitor a specific killing effect on therapy-induced senescent tumor cells, thereby completing the present invention.

### SUMMARY OF THE INVENTION

An object of the present invention is to provide a composition for killing senescent cells, comprising a Pim-1 kinase inhibitor.

Another object of the present invention is to provide a pharmaceutical composition for preventing or treating a tumor comprising a Pim-1 kinase inhibitor and a method for administering the same.

Still another object of the present invention is to provide the use of a Pim-1 kinase inhibitor for killing senescent cells.

Yet another object of the present invention is to provide the use of a Pim-1 kinase inhibitor for preventing or treating a tumor.

Still yet another object of the present invention is to provide the use of a Pim-1 kinase inhibitor for manufacturing a composition for killing senescent cells.

A further object of the present invention is to provide the use of a Pim-1 kinase inhibitor for preparing a pharmaceutical composition for preventing or treating a tumor.

Another further object of the present invention is to provide a method of killing senescent cells using a Pim-1 kinase inhibitor, and a method of preventing or treating a tumor a Pim-1 kinase inhibitor.

To achieve the above objects, the present invention provides a composition for killing senescent cells, comprising a Pim-1 kinase inhibitor.

The present invention also provides the use of a Pim-1 kinase inhibitor for manufacturing a composition for killing senescent cells.

The present invention also provides the use of a Pim-1 kinase inhibitor for killing senescent cells.

The present invention also provides a method for killing senescent cells, comprising a step of administering a Pim-1 kinase inhibitor.

The present invention also provides a pharmaceutical composition for preventing or treating senescence-related disease or age-related disease, comprising a Pim-1 kinase inhibitor.

The present invention also provides the use of a Pim-1 kinase inhibitor for preventing or treating senescence-related disease or age-related disease or for preparing a pharmaceutical composition therefor.

The present invention also provides a method for preventing or treating senescence-related disease or age-related disease, comprising a step of administering a Pim-1 kinase inhibitor.

The present invention also provides a pharmaceutical composition for preventing or treating a tumor, comprising a Pim-1 kinase inhibitor, which is administered after tumor cell senescence-inducing therapy.

The present invention also provides the use of a Pim-1 kinase inhibitor for preparing a pharmaceutical composition for preventing or treating a tumor, which is administered after tumor cell senescence-inducing therapy.

The present invention also provides a method for preventing or treating a tumor, comprising steps of: inducing senescence of tumor cells; and killing the senescent tumor cells by administering a Pim-1 kinase inhibitor.

The present invention also provides a method for preventing or treating a tumor, comprising steps of: detecting senescent cells in a sample isolated from a subject; and if the senescent cells are detected, administering a Pim-1 kinase inhibitor to the subject.

### BRIEF DESCRIPTION OF DRAWINGS

FIG. 1 shows the increase in cell size and SA-β-gal staining results (1a), the proportion of SA-β-gal positive cells (1b, left), and the level of p21 mRNA expression (1b, right) in each cancer cell line after causing therapy-induced senescence (TIS) by treatment of breast cancer cell lines (MCF7, ZR75-1, Hs578T, and MDA-MB-231) with 100 nM of adriamycin.

FIG. 2a is a Venn diagram showing the changes in mRNA expression levels in MCF7 and MDA-MB-231, confirmed by next-generation sequencing (NGS) after therapy-induced senescence (TIS).

FIG. 2b is a volcano plot showing the change in expression of each gene in each cell line upon TIS for 28 genes, which are targeted by FDA-approved drugs, selected among genes whose expression was statistically significantly increased in common in MCF7 and MDA-MB-231 cell lines, after therapy-induced senescence before therapy-induced senescence.

FIG. 2c shows the results of analyzing the changes in the expression of the 28 previously selected genes after therapy-induced senescence of other breast cancer cell lines (BT474, Hs578T, T47D, and HCC1937) that were not subjected to RNAseq.

FIG. 3a shows the results of CCK8 assay performed to evaluate the cell division inhibition and apoptosis of control (Ctrl) cells and therapy-induced senescent cells (TIS) after treating MCF7 control cells and TIS cells with varying concentrations of inhibitors (small-molecule compounds excluding antibodies) targeting the final selected genes.

FIG. 3b shows the results of CCK8 assay indicating that cell viability was significantly inhibited in a therapy-induced senescent cell group compared to a control group after treating the MCF7 cell line with a PIM1 kinase inhibitor (PIM1/2 kinase inhibitor VI, Calbiochem, CAS 587852-28-6) and ABT-263, which is known as a senolytic drug that selectively kills senescent cells.

FIG. 3c shows the results of microscopy performed to evaluate the cell killing effect of PIM1/2 kinase inhibitor VI after treating control MCF7 cells and therapy-induced senescent cells with various concentrations of PIM1/2 kinase inhibitor VI (Calbiochem, CAS 587852-28-6).

FIGS. 3d and 3e show the results of CCK8 assay and microscopy performed to evaluate the cell killing effect of SGI-1776 after treating MCF7 cells (Ctrl and TIS) with another PIM1 kinase inhibitor, SGI-1776 (Selleckhem, Catalog No. S2198, CAS 1025065-69-3).

FIG. 4 shows the results of analyzing the expression of PIM1 kinase protein in various cancer cell lines (MKN1, MKN74, SNU601: gastric cancer, SNU478, SNU1196, SNU869: biliary tract cancer, Hct116, SW480: colon cancer, BxPC3: pancreatic cancer, HeLa S3: cervical cancer, PC3: prostate cancer, and HSC4: head and neck cancer) in addition to breast cancer cells after causing therapy-induced senescence (TIS).

FIG. 4a shows the results of analyzing the changes in mRNA expression of PIM kinase family genes, including PIM1, in the indicated cancer cell lines after causing therapy-induced senescence by adriamycin.

FIG. 4b shows the results of analyzing the quantitative changes of the PIM1 protein in various cell lines under the same conditions.

FIG. 4c shows the results of analyzing the change in mRNA expression of PIM1 kinase in various cancer cells after causing therapy-induced senescence (TIS) by treatment with cisplatin.

FIGS. 5a and 5b show the results of microscopy and CCK8 assay performed to evaluate the cell killing effect of PIM 1/2 inhibitor VI on control (Ctrl) cells and therapy-induced senescent (TIS) cells after treating cancer cell lines (HSC4: head and neck cancer, SNU601: gastric cancer, and SW480: colon cancer) with PIM 1/2 inhibitor VI.

FIG. 6a shows the change in expression of PIM1 kinase protein 48 hours after treatment of MCF7 cells with siRNA targeting the Pim1 kinase gene.

FIGS. 6b and 6c show the results of microscopy and CCK8 assay performed to evaluate the cell killing effect of siRNA on control (Ctrl) cells and therapy-induced senescent (TIS) cells (TIS) after inhibiting the expression of PIM1 kinase protein by treatment with various concentrations of siRNA targeting the Pim1 kinase gene for 48 hours.

### MODE FOR THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

Senescence of tumor cells is used as a major mechanism of various anticancer therapies for the treatment of tumors, such as chemotherapy and radiotherapy. SASP of senescent cells has been report to primarily exhibit antitumor effects, but in the long term, senescent cells may exhibit pro-tumor effects, thereby promoting tumor formation, recurrence, and metastasis and causing various side effects. From this perspective, “one-two punch” therapy, which prevents long-term side effects by selectively killing therapy-induced senescent cells (TIS cells) by administration of senolytic drugs after primary treatment of tumors by induction of senescence, has recently been gaining attention, but the types of reported senolytic drug are significantly lacking.

In one Example of the present invention, as a result of analyzing the gene expression profiles of therapy-induced senescent cells (TIS cells) and analyzing the senolytic effects based thereon, it was confirmed that the expression of Pim-1 kinase was significantly increased in therapy-induced senescent cells (TIS cells), and that an activity inhibitor of Pim-1 kinase or inhibition of the expression of the gene exhibited high selective toxicity to therapy-induced senescent cells.

Therefore, in one aspect, the present invention provides a composition for killing senescent cells, comprising a Pim-1 kinase inhibitor.

In the present invention, the term “Pim-1 kinase” refers to a serine/threonine protein kinase encoded by the PIM-1 kinase gene. Pim-1 kinase is known to be involved in pathways such as regulation of MYC transcriptional activity, regulation of cell cycle progression, apoptosis, and transcriptional activation. Pim-1 kinase is also well known as an oncogene (The International Journal of Biochemistry & Cell Biology. 37 (4): 726-30).

In the present invention, the term “Pim-1 kinase inhibitor” is used to mean any chemical or biological agent capable of inhibiting the expression of Pim-1 kinase gene or the activity of Pim-1 kinase protein.

In the present invention, the Pim-1 kinase inhibitor may be a Pim-1 kinase gene expression inhibitor or a PIM1 kinase protein activity inhibitor.

In the present invention, the term “gene expression inhibitor” means any agent that inhibits or suppresses the expression by transcription and translation of a target gene. The term may include any type of agent that specifically inhibits expression of a target gene, and includes both an inhibitor that regulates the transcription stage and an inhibitor that regulates post-transcriptional translation, such as RNAi. Examples of the Pim-1 kinase gene expression inhibitor include, but are not limited to, antisense nucleotides, siRNAs (small interfering RNAs), shRNAs (small hairpin RNAs), ribozymes, aptamers, anti-microRNAs, microRNA mimics, nucleases such as zinc finger nucleases (ZFNs) and transcription activator-like effector nucleases (TALENs), CRISPR-Cas9 agents (e.g., gRNA, sgRNA), polycistronic tRNA-gRNA system agents, and self-ribozyme-flanked RNAs, which target the PIM-1 kinase gene (Gao & Zhao 2014, Xie et al. 2015, Zetsche et al. 2017).

In the present invention, the Pim-1 kinase gene expression inhibitor may preferably be an siRNA targeting the Pim-1 kinase gene. More preferably, the siRNA may comprise a sequence set forth in SEQ ID NO: 1, without being limited thereto.

The antisense nucleotide binds (hybridizes) to the complementary nucleotide sequence of DNA, immature-mRNA, or mature mRNA, thereby disrupting the flow of genetic information from DNA to protein. The siRNA has a similar working principle to the antisense oligonucleotide, but it is a double strand of 21 to 25 nucleotides, and destroys mRNA through enzyme complexes called RISCs (RNA-induced silencing complexes) rather than RNase H. The CRISPR-Cas9 (CRISPR gene scissors) acts as a restriction enzyme that recognizes a specific nucleotide sequence and cuts the DNA at that site.

In the present invention, the term “protein activity inhibitor” refers to any agent that inhibits or suppresses the activity of a target protein. The protein activity inhibitor may directly inhibit the activity of a target protein, or interfere with the interaction of the target protein with other proteins, thereby inhibiting its function and blocking a downstream signaling pathway. Examples of the Pim-1 kinase protein activity inhibitor include, but are not limited to, compounds, peptides, peptidomimetics, substrate analogs, aptamers, and antibodies, which specifically bind to Pim-1 kinase protein.

In the present invention, the Pim-1 kinase inhibitor used may be selected, without limitation, from various Pim-1 kinase inhibitors known in the art.

In the present invention, the compound as the Pim-1 kinase inhibitor includes any compound capable of inhibiting the activity of Pim-1 kinase, which is a target protein. Examples of the Pim-1 kinase inhibitor include, but are not limited to, a flavonoid inhibitor, isoxazolo[3,4-b]quinoline-3,4(1H,9H)-dione, ETP-45299, an imidazopyridazine-based compound (SGI-1776), a thiazolidinedione-based compound (AZD1208), a fluoropicolinamidine-based compound (PIM447), PIM1/2 kinase inhibitor VI, and the like.

In the present invention, the aptamer is a single-stranded DNA or RNA molecule, and may be obtained by isolating an oligomer, which binds to a specific chemical molecule or biological molecule with high affinity and selectivity, by an evolutionary method using an oligonucleotide library called SELEX (systematic evolution of ligands by exponential enrichment). The aptamer can specifically bind to a target protein and modulate the activity of the target protein. For example, it can block the activity of the target protein by binding thereto. The antibody refers to a collection of antibody protein molecules comprising one or more complementarity determining regions, an antibody protein molecule, a binding fragment, or a derivative thereof.

In the present invention, the term “inhibitor” may be used interchangeably with the term “antagonist” or “suppressor”.

In the present invention, the terms “target” used in the terms “target protein”, “target gene”, “targeting”, etc. means a target whose activity is to be regulated, and particularly, in the present invention, the inhibitor is characterized by being able to regulate the expression or activity of a target gene. In the present invention, the target protein is Pim-1 kinase.

In the present invention, the term “senescence” refers to the progression from an actively dividing cell to a metabolically active, non-dividing cell. In the present invention, the term “senescent cell” is used as a concept that includes intermediate senescent cells in the senescence process and fully senescent cells whose division has been completely inactivated.

In the present invention, the senescent cells may exhibit the following characteristics, without being limited thereto:
i) inhibition of growth;
ii) inhibition of re-entry into the cell cycle even after treatment with physiological mitogens;
iii) resistance to apoptosis; and/or
iv) acquisition of differentiated functions.

In the present invention, senescence may be classified, in a non-limiting manner, as follows.

“Replicative cellular senescence” refers to the limitation of the number of cell divisions of differentiated cells due to telomere deterioration.

“Premature cellular senescence” refers to cell cycle arrest caused by various stresses such as UV, reactive oxygen species, chemotherapy, environmental toxins, ionizing radiation, smoking, and oncogenic mutations without telomere deterioration. “Oncogene-induced senescence” refers to an example of premature cellular senescence, which is cellular senescence caused by increased expression or mutation of oncogenes.

“Therapy-induced senescence” means that cells are forced into a senescent state by therapeutic agents administered to treat certain diseases. Typically, therapy-induced senescence occurs due to the administration of anticancer agents or anticancer therapy for the treatment of tumors. From an oncological perspective, therapy-induced senescence is generally known to be caused by various anticancer therapies such as chemotherapy, biological therapy, and radiotherapy (Nature Reviews Cancer volume 22, pages 340-355 (2022)).

In the present invention, the senescent cell may be a cell whose growth and/or division have/has ceased. In the present invention, the senescent cell may be characterized by maintaining metabolic activity. In the present invention, the senescent cell may be a cell whose growth and/or division have/has ceased permanently, without being limited thereto, and may re-enter the division cycle due to external factors.

In the present invention, the senescent cells may be found in or isolated from regenerative tissues, including vascular system, hematopoietic system, epithelial organs, and stroma.

In the present invention, the senescent cells may be found in or isolated from a senescence-related lesion site. Examples of the senescence-related lesion site include, but are not limited to, chronic age-related disease, inflammatory disease, osteoarthritis, arteriosclerosis, immune disease, neoplastic disease, and pre-tumor or tumor tissues.

In one Example of the present invention, cancer cells were induced to senescence by treatment with adriamycin, and senescent cells were isolated through SA-b-gal staining and p21 mRNA expression levels. Therefore, in the present invention, the senescent cells may be senescent cancer cells.

In the present invention, the cancer may be, for example, breast cancer, gastric cancer, colorectal cancer, head and neck cancer, cervical cancer, bile duct cancer, lung cancer, or pancreatic cancer, without being limited thereto.

In the present invention, the senescent cells may preferably be oncogene-induced senescence cells (OIS cells) or therapy-induced senescent cells (TIS cells), more preferably therapy-induced senescent cells (TIS cell).

In the present invention, the therapy-induced senescent cells may be cells induced to senescence by chemotherapy, biological therapy, and/or radiotherapy.

In the present invention, the therapy-induced senescent cells may be therapy-induced senescent cancer cells or normal cells.

The composition for killing senescent cells according to the present invention may be prepared in a unit dose form or prepared to be contained in a multi-dose container by formulating with one or more carriers or excipients.

The term “carrier” as used in the present invention may mean a carrier or diluent that does not stimulate a living organism and does not abrogate the biological activity and properties of a compound to be injected. The type of carrier that may be used in the present invention is not particularly limited, and any carrier commonly used in the art may be used. Non-limiting examples of the carrier include saline, sterile water, Ringer's solution, buffered saline, albumin injection solution, dextrose solution, maltodextrin solution, glycerol, ethanol, etc. These may be used alone or in combination of two or more. The carrier may include a non-naturally occurring carrier. In addition, if necessary, other conventional additives such as antioxidants, buffers, and/or bacteriostatic agents may be added and used, and diluents, dispersants, surfactants, binders, lubricants, etc. may be additionally added and used to prepare injectable formulations such as aqueous solutions, suspensions, and emulsions, pills, capsules, granules, or tablets.

The composition is formulated with commonly used diluents or excipients, such as fillers, extenders, binders, wetting agents, disintegrants, and surfactants.

The composition for killing senescent cells according to the present invention may be formulated and used in various forms according to conventional methods. Suitable formulations include, but are not limited to, oral formulations such as tablets, pills, powders, granules, sugar-coated tablets, hard or soft capsules, solutions, suspensions or emulsions, injections, or aerosols, external preparations, suppositories, and sterile injectable solutions.

The composition for killing senescent cells according to the present invention may be prepared into a suitable formulation using a pharmaceutically inactive organic or inorganic carrier. That is, if the formulation is a tablet, a coated tablet, a sugar-coated tablet, or a hard capsule, it may contain lactose, sucrose, starch or derivatives thereof, talc, calcium carbonate, gelatin, stearic acid or salts thereof, etc. In addition, if the formulation is a soft capsule, it may contain vegetable oils, waxes, fats, semi-solid and liquid polyols, etc. In addition, if the formulation is in the form of a solution or syrup, it may contain water, polyols, glycerol, and vegetable oils.

The composition for killing senescent cells according to the present invention may further contain, in addition to the carrier described above, a preservative, a stabilizer, a wetting agent, an emulsifier, a solubilizer, a sweetener, a coloring agent, an osmotic pressure regulator, an antioxidant, etc.

In the present invention, the term “administration” means introducing the composition of the present invention to a subject by any suitable method. The pharmaceutical composition of the present invention may be administered orally or parenterally through various routes as long as it can reach a target tissue. The method of administration of the pharmaceutical composition according to the present invention is not particularly limited and conventional methods used in the art may be employed. Regarding the method of administration, the pharmaceutical composition may be administered in a conventional manner, for example, via intravenous, intraarterial, intraperitoneal, intramuscular, intraarterial, intraperitoneal, intrasternal, transdermal, intranasal, inhalation, topical, intrarectal, oral, intraocular or intradermal routes, without being limited to the above examples.

The composition for killing senescent cells according to the present invention is determined depending on the type of drug as an active ingredient, along with various related factors such as the purpose of administration, the route of administration route, the subject's age, sex and body weight, and the severity of the disease.

In the present invention, the senescent cells may be positive to SA-b-gal staining. In the present invention, the senescent cells may have increased expression of p21.

In one example of the present invention, genes with increased expression were identified by analyzing gene expression of therapy-induced aged tumor cells.

In the present invention, the senescent cells may have increased expression of any one or more genes selected from the group consisting of ATP2B4, CPE, CXCR4, DGAT2, F2R, FYN, ICAM1, IL6, INSR, KDM7A, MAOA, MAP2, MAST4, PDE4B, PIM1, PINK1, PLD1, PSMB9, QPRT, S1PR3, SLC6A8, UGCG and VEGFA compared to that in normal control cells.

In the present invention, the senescent cells preferably have increased expression of the PIM1 gene compared to that in normal control cells.

The composition for killing senescent cells according to the present invention may selectively kill senescent cells, preferably senescent cells with increased expression of Pim-1 kinase.

In the present invention, the composition for killing senescent cells may exhibit the effect of killing senescent cells, preferably the effect of selectively killing senescent cells.

The killing of senescent cells may be performed for the prevention or treatment of various diseases related to senescence. Therefore, the composition for killing senescent cells according to the present invention may be used for the prevention or treatment of diseases related to senescence. For example, the composition for killing senescent cells according to the present invention may selectively kill senescent cells, thereby strengthening the immune system, extending lifespan, and improving the quality of life of a subject.

In the present invention, the senescence-related disease is meant to include any disease or condition that is totally or partially mediated by the induction or maintenance of a non-proliferative or senescent condition in a cell or cell population (e.g., tissue). Senescence-related diseases include, for example, senescence-related diseases that are not visible, degeneration of tissues or organs, and visible diseases such as degenerative diseases or hypofunction disorders. More specific examples include, but are not limited to, Alzheimer's disease, Parkinson's disease, cataracts, macular degeneration, glaucoma, arteriosclerosis, acute coronary syndrome, myocardial infarction, stroke, hypertension, idiopathic pulmonary fibrosis (IPF), chronic obstructive pulmonary disease (COPD), osteoarthritis, type 1 or type 2 diabetes, obesity, cancer, tumors, adipose dysfunction, coronary artery disease, cerebrovascular disease, periodontal disease, atrophy in various tissue, cancer therapy-related disorders such as fibrosis, brain and heart damage, myelodysplastic syndrome, etc. Age-related diseases include, for example, but are not limited to, premature aging diseases such as Hutchinson-Gilford progeria syndrome, Werner syndrome, Bloom syndrome, Rothmund-Thomson syndrome, Cockayne syndrome, xeroderma pigmentosum, trichothiodystrophy, combined xeroderma pigmentosum-Cockayne syndrome, restrictive dermopathy, ataxia telangiectasia, Fanconi anemia, Friedreich's ataxia, dyskeratosis congenital, aplastic anemia, IPF, and the like. In the present invention, senescence-related diseases may be identified by detecting the presence or absence of senescent cells.

In the present invention, the senescence-related disease may be cancer or a tumor, and more preferably, may be senescent cancer or a senescent tumor.

In the present invention, the senescence-related disease may be diagnosed by detecting senescent cells. The diagnosis of the senescence-related disease may be performed by detection of DNA replication deficiency by incorporation of BrdU or 3H-thymidine, immunostaining for proteins such as proliferating cell nuclear antigen (PCNA) and Ki-67, SA-β-gal histochemical staining, detection of expression of p16, p19, Pail, Igfbp2, IL-6, Mmp13, Nrg1, DEC1 (differentiated embryo-chondrocyte expressed-1), p15, CDK1, and DCR2 (decoy death receptor-2), or detection of senescence-associated heterochromatin foci (SAHFs) and/or senescence-associated DNA-damage foci (SDFs). The SAHFs may be detected by preferential binding of DNA dyes such as DAPI and the presence of histone modifications associated with heterochromatin, such as H3 Lys 9 methylation and/or heterochromatin protein 1 (HP1). As another example, senescent cells may be detected by methods such as those described in U.S. Pat. No. 5,491,069 and U.S. Patent Application No. 2010/0086941, without being limited thereto. In the present invention, the composition for killing senescent cells may be prepared in the form of a pharmaceutical composition, a food composition, a food supplement composition, a cosmetic composition, or a cosmetic.

In another aspect, the present invention relates to a pharmaceutical composition for preventing or treating senescence-related disease or age-related disease, comprising a Pim-1 kinase inhibitor.

In another aspect, the present invention relates to the use of a Pim-1 kinase inhibitor for preventing or treating senescence-related disease or age-related disease or the use of the Pim-1 kinase inhibitor for preparing a pharmaceutical composition therefor.

In another aspect, the present invention relates to a method for preventing or treating senescence-related disease or age-related disease, comprising a step of administering a Pim-1 kinase inhibitor.

In another aspect, the present invention relates to senolytics comprising a Pim-1 kinase inhibitor.

In the present invention, the term “senolytics” or “senolytic drugs” refers to an agent or pharmaceutical composition capable of selectively killing senescent cells.

In another aspect, the present invention relates to the use of a Pim-1 kinase inhibitor for killing senescent cells.

In another aspect, the present invention relates to the use of a Pim-1 kinase inhibitor for manufacturing a composition for killing senescent cells.

In another aspect, the present invention relates to a method for killing senescent cells or a method for selectively killing senescent cells, comprising a step of administering a Pim-1 kinase inhibitor to a subject.

In the present invention, the subject may be administered an anticancer agent or treated with radiotherapy to induce tumor senescence, prior to administration of the Pim-1 kinase inhibitor.

In the present invention, the term “selective killing of senescent cells” means targeting senescent cells, causing the cells to reach a state of cell death. In the present invention, the selective killing is used to mean not only specifically killing only senescent cells, but also having a significantly superior cell killing effect on senescent cells compared to non-senescent cells.

Unless otherwise stated, in the above aspects of the present invention, each component and term may have the same characteristics as described above with respect to the composition for killing senescent cells.

In one Example, it was confirmed that, when tumor cells induced to senescence by a chemotherapeutic agent was treated with a Pim-1 kinase activity inhibitor (PIM1/2 kinase inhibitor VI) or with siRNA targeting the PIM-1 kinase gene, a significant killing effect was observed on the therapy-induced senescent tumor cells compared to normal tumor cells, indicating that the therapy-induced senescent cells could be selectively killed.

The Pim-1 kinase inhibitor of the present invention and the composition for killing senescent cells comprising the same may be used as a senolytic drug for “one-two punch therapy” that induces senescence of tumor cells by primary anticancer therapy, and then kills senescent tumor cells, thereby preventing long-term tumor recurrence and side effects.

Therefore, in another aspect, the present invention relates to a pharmaceutical composition for preventing or treating a tumor, comprising a Pim-1 kinase inhibitor, which is administered after tumor cell senescence-inducing therapy.

Since Pim-1 was identified as an oncogene, various Pim-1 kinase inhibitors have been developed to treat tumors, and combination therapy with anticancer agents such as doxorubicin has also been reported (Jeffrey D. Altenburg et al. Blood (2014) 124 (21) 4505). However, since Pim-1 kinase is known to be involved in oncogene-induced senescence, it has been reported that the inhibition of expression of Pim-1 kinase can induce tumor formation in normal cells, and senescence of tumor cells induced by Pim-1 kinase can be helpful in the treatment of tumors in the long term, and that inhibition of Pim-1 kinase can induce tumor formation and proliferation (Bo Jin et al. Aging cell 2014, 13, pp. 879-889; Wu et al., Med Sci Monit, 2019, 25: 8651-8659). The above characteristics of Pim-1 kinase mean that Pim-1 kinase inhibitors may have reduced efficacy or show unexpected side effects when used as monotherapy for the treatment of tumors or in combination with other anticancer therapies, radiotherapy, etc. that induce cell senescence.

Therefore, the pharmaceutical composition of the present invention may be administered after tumor cell senescence-inducing therapy.

The pharmaceutical composition of the present invention may be used as a senolytic drug that selectively kills therapy-induced senescent tumor cells formed by anticancer therapy that induces senescence, in one-two punch therapy for the treatment of tumors.

In the present invention, the term “one-two punch therapy” refers to therapy in which primary treatment is performed using conventional anticancer therapy for inducing tumor senescence, such as chemotherapy, biological agents, radiotherapy, etc., and then a senolytic drug that selectively kills senescent cells is administered, thereby selectively killing therapy-induced senescent tumor cells formed by the treatment. Since “one-two punch” therapy is not based on co-administration of drugs, but is based on sequential treatment using each drug, it can avoid combination toxicity caused by a combination of drugs and prevent long-term side effects caused by therapy-induced senescent tumor cells.

In the present invention, the Pim-1 kinase inhibitor may be a Pim-1 kinase gene expression inhibitor or a PIM1 kinase protein activity inhibitor.

In the present invention, examples of the Pim-1 kinase gene expression inhibitor include, but are not limited to, antisense nucleotides, siRNAs (small interfering RNAs), shRNAs (small hairpin RNAs), ribozymes, aptamers, anti-microRNAs, microRNA mimics, nucleases such as zinc finger nucleases (ZFNs) and transcription activator-like effector nucleases (TALENs), CRISPR-Cas9 agents (e.g., gRNA, sgRNA), polycistronic tRNA-gRNA system agents, and self-ribozyme-flanked RNAs, which target the PIM-1 kinase gene (Gao & Zhao 2014, Xie et al. 2015, Zetsche et al. 2017).

In the present invention, the Pim-1 kinase gene expression inhibitor may preferably be an siRNA targeting the Pim-1 kinase gene. More preferably, the siRNA may comprise a sequence set forth in SEQ ID NO: 1, without being limited thereto.

In the present invention, the protein activity inhibitor is able to directly inhibit the activity of a target protein, or interfere with the interaction of the target protein with other proteins, thereby inhibiting its function and blocking a downstream signaling pathway. Examples of the Pim-1 kinase protein activity inhibitor include, but are not limited to, compounds, peptides, peptidomimetics, substrate analogs, aptamers, and antibodies, which specifically bind to Pim-1 kinase protein.

In the present invention, the Pim-1 kinase inhibitor used may be selected, without limitation, from various Pim-1 kinase inhibitors known in the art.

In the present invention, the compound as the Pim-1 kinase inhibitor includes any compound capable of inhibiting the activity of Pim-1 kinase, which is a target protein. Examples of the Pim-1 kinase inhibitor include, but are not limited to, a flavonoid inhibitor, isoxazolo[3,4-b]quinoline-3,4(1H,9H)-dione, ETP-45299, an imidazopyridazine-based compound (SGI-1776), a thiazolidinedione-based compound (AZD1208), a fluoropicolinamidine-based compound (PIM447), PIM1/2 kinase inhibitor VI, and the like.

The term “tumor cell senescence-inducing therapy” of the present invention refers to anticancer therapy that induces senescence of tumor cells to inhibit growth and proliferation of the tumor cells and has effects such as arresting adjacent cancer cells by SASP secreted from senescent cells and recruiting immune cells. In the art to which the present invention pertains, conventional anticancer therapy, such as conventional chemotherapy and radiotherapy, is known to induce senescence of tumor cells (Faget, D. V. et al., Nat. Rev. Cancer 19, 439-453 (2019); Krizhanovsky, V. et al. Cell 134, 657-667 (2008); Ritschka, B. et al., Genes Dev. 31, 172-183 (2017); Chang, B. D. et al. Oncogene 18, 4808-4818 (1999)), and senescence induced by this anticancer therapy is referred to as “therapy-induced senescence”.

In the present invention, the tumor cell senescence-inducing therapy may induce therapy-induced senescence of tumor cells.

In the present invention, the senescence-inducing therapy may administration of an anticancer agent or radiotherapy.

In the present invention, the anticancer agent may be a chemotherapeutic agent.

In the present invention, the anticancer agent may preferably be a chemical or biological substance known to prevent or treat cancer.

In the present invention, the anticancer agent may be selected from the group consisting of actinomycin D, adriamycin, amsacrine, ara-C, 9-3-D-arabinosyl-2-fluoroadenine, BCNU, bleomycin, camptothecin, carboplatin, 2-chloro-2-deoxyadenosine, CPT-11, cyclophosphamide, docetaxel, doxorubicin, edotecarin, etoposide, fludarabine, 5-fluorouracil (5-FU), gemcitabine, HU-gemzar, irinotecan, methotrexate, 6-mercaptopurine, mitomycin-C, paclitaxel, cis-platin, SN-38, taxol, thiotepa, 6-thioguanine, trimetrexate, vinblastine, vincristine, and VP-16, without being limited thereto.

In the present invention, the anticancer agent may be a DNA-damaging agent. The DNA-damaging agent may be selected from the group consisting of, for example, alkylating agents, antimetabolites, metabolic antagonists, platinum analogues, topoisomerase I inhibitors, and topoisomerase II inhibitors, without being limited thereto.

In the present invention, the anticancer agent may be a mitotic inhibitor.

In the present invention, the anticancer agent may be a targeted anticancer agent. Examples of the targeted anticancer agent include, but are not limited to, CDC7 inhibitors (e.g., XL413, TAK-931), telomerase inhibitors (e.g., GRN163L, BIBR15), epigenetic regulators (e.g., decitabine, vorinostat), CDK inhibitors (e.g., palbociclib, abemaciclib, ribociclib, PF-06873600), AURK inhibitors (e.g., alisertib, barasertib), PLK1 inhibitors (e.g., BI-2535, BI-6727), MDM2-p53 disruptors (e.g., Nitlin3, RG7112, UBX0101), PTEN inhibitors (e.g., VO-OHpic), and the like.

In the present invention, the anticancer agent may include an anti-hormonal agent, an aromatase inhibitor, an antiandrogen agent, a protein kinase inhibitor, an antisense oligonucleotide, a ribozyme, a tumor vaccine, etc.

In the present invention, the anticancer agent may be a therapeutic antibody-drug conjugate. Examples of the therapeutic antibody include, but are not limited to, alemtuzumab (Campath), bevacizumab (AVASTIN, Genentech), cetuximab (Imclone), panitumumab (Amgen), rituximab (Genentech/Biogen Idec), pertuzumab (OMNITARG™, 2C4, Genentech), trastuzumab (HERCEPTIN, Genentech), and tositumomab (Corixia).

The terms “alkylating agent”, “metabolic antagonist”, “antitumor antibiotic”, “platinum analogue”, “topoisomerase I inhibitor”, “topoisomerase II inhibitor”, “mitotic inhibitor”, “targeted anticancer agent”, and “therapeutic antibody”, which are exemplarily listed in the present invention, are meant to include, without limitation, any clinically used chemical or biological anticancer agent that induces cellular senescence.

The pharmaceutical composition of the present invention is able to selectively kill therapy-induced senescent tumor cells induced by tumor cell senescence-inducing therapy.

In the present invention, the therapy-induced senescent tumor cells may be positive to SA-b-gal staining. In the present invention, the senescent cells may have increased expression of p21.

In one Example of the present invention, genes with increased expression were identified by analyzing gene expression of therapy-induced aged tumor cells.

In the present invention, the therapy-induced senescent cells may have increased expression of any one or more genes selected from the group consisting of ATP2B4, CPE, CXCR4, DGAT2, F2R, FYN, ICAM1, IL6, INSR, KDM7A, MAOA, MAP2, MAST4, PDE4B, PIM1, PINK1, PLD1, PSMB9, QPRT, S1PR3, SLC6A8, UGCG and VEGFA compared to that in normal control cells.

In the present invention, the therapy-induced senescent cells may preferably have increased expression of the PIM1 gene compared to that in normal control cells.

The pharmaceutical composition of the present invention may be administered sequentially after tumor cell senescence-inducing therapy, preferably after administration of an anticancer agent and/or radiotherapy.

The pharmaceutical composition of the present invention may be administered at a certain time after tumor cell senescence-inducing therapy is applied to a subject. In the present invention, the pharmaceutical composition may preferably be administered after the senescent cells induced by tumor cell senescence-inducing therapy are sufficiently formed.

In the present invention, the pharmaceutical composition may preferably be administered at several hours, several days, several weeks, several months, or several years after the senescence-inducing therapy, and more preferably, it may be administered at about 1 hour to about 24 hours, about 1 day to about 6 days, about 1 week to about 4 weeks, about 1 month to 11 months, or about 1 year or more after the senescence-inducing therapy, without being limited thereto. For application of a more desirable “one-two punch” therapy, the pharmaceutical composition may be administered after detecting the presence of senescent cells in a sample isolated from a subject.

In the present invention, the pharmaceutical composition may further comprise another senolytic drug in addition to the active ingredient, Pim-1 kinase inhibitor. Senolytic drugs used for the treatment of cancer or tumors are well known in the art (Wang, L., Lankhorst, L. & Bernards, R. Exploiting senescence for the treatment of cancer. Nat Rev Cancer 22, 340-355 (2022)).

In another aspect, the present invention relates to a method for administering the pharmaceutical composition, comprising a step of administering a Pim-1 kinase inhibitor after tumor cell senescence-inducing therapy.

In another aspect, the present invention relates to the use of a Pim-1 kinase inhibitor for preparing a pharmaceutical composition for preventing or treating a tumor comprising the Pim-1 kinase inhibitor, wherein the pharmaceutical composition may be administered after tumor cell senescence-inducing therapy.

In another aspect, the present invention relates to the use of a Pim-1 kinase inhibitor as a senolytic drug in a “one-two punch” therapy for the prevention or treatment of tumors.

In another aspect, the present invention relates to a method for preventing or treating a tumor, comprising steps of: inducing senescence of tumor cells; and killing the senescent tumor cells by administering a Pim-1 kinase inhibitor.

In the present invention, the step of inducing senescence of tumor cells may be performed through tumor cell senescence-inducing therapy for the subject.

In the present invention, it can be easily understood by those skilled in the art that the tumor cell senescence-inducing therapy may share the same characteristics as those described above with respect to the pharmaceutical composition, unless stated otherwise.

In the present invention, the Pim-1 kinase inhibitor may be administered as a direct Pim-1 kinase dilution, or may be prepared and administered as a composition for killing senescent cells or a pharmaceutical composition of the present invention.

In the present invention, in addition to sequential administration of tumor cell senescence-inducing therapy and the Pim-1 kinase inhibitor, tumor cells may be treated through the death of senescent cells present in the subject.

Therefore, in another aspect, the present invention relates to a method for preventing or treating a tumor, comprising steps of: detecting senescent cells in a subject or a sample isolated from the subject; and if the senescent cells are detected, administering a Pim-1 kinase inhibitor to the subject.

In the present invention, the step of detecting the senescent cells may be performed in vivo or in vitro. In the present invention, the step of detecting the senescent cells may comprise detecting an increase in cell size or an increase in cell number visually or through a microscope.

In the present invention, the step of detecting the senescent cells may be performed by detecting various senescent cell markers known in the art. For example, the step of detecting the senescent cells may be performed by detection of DNA replication deficiency by incorporation of BrdU or 3H-thymidine, immunostaining for proteins such as proliferating cell nuclear antigen (PCNA) and Ki-67, SA-β-gal histochemical staining, detection of expression of p16, p19, Pail, Igfbp2, IL-6, Mmp13, Nrg1, DEC1 (differentiated embryo-chondrocyte expressed-1), p15, CDK1, and DCR2 (decoy death receptor-2), or detection of senescence-associated heterochromatin foci (SAHFs) and/or senescence-associated DNA-damage foci (SDFs). The SAHFs may be detected by preferential binding of DNA dyes such as DAPI and the presence of histone modifications associated with heterochromatin, such as H3 Lys 9 methylation and/or heterochromatin protein 1 (HP1). As another example, senescent cells may be detected by methods such as those described in U.S. Pat. No. 5,491,069 and U.S. Patent Application No. 2010/0086941, without being limited thereto.

In the present invention, the step of detecting the senescent cells may be performed by analyzing the expression level of any one or more genes selected from the group consisting of ATP2B4, CPE, CXCR4, DGAT2, F2R, FYN, ICAM1, IL6, INSR, KDM7A, MAOA, MAP2, MAST4, PDE4B, PIM1, PINK1, PLD1, PSMB9, QPRT, S1PR3, SLC6A8, UGCG and VEGFA. In the present invention, cells having an increased expression level of any one or more genes selected from the above group increases compared to that in normal control cells may be detected as the senescent cells.

In the present invention, the step of detecting the senescent cells may comprise a step of analyzing the expression level of the PIM1 gene.

In the present invention, cells having an increased expression level of the PIM-1 kinase gene compared to that in normal control cells may be detected the senescent cells.

In the present invention, if senescent cells are detected in the subject or the sample isolated from the subject, the PIM-1 kinase inhibitor may be administered.

In the present invention, the PIM-1 kinase inhibitor may be administered by the same administration route and administration method as those described above with respect to the composition or pharmaceutical composition for killing senescent cells.

In the present invention, the term “prevention” may mean any action that inhibits or delays the onset of a tumor by administering the composition or pharmaceutical composition for killing senescent cells according to the present invention to a subject suspected of having the disease.

In the present invention, the term “treatment” may mean any action that alleviates or beneficially changes the symptoms of a disease by administering the composition or pharmaceutical composition for killing senescent cells according to the present invention to a subject suffering from the disease.

The term “individual” or “subject” as used in the present invention may mean all animals, including humans, that have or are likely to develop a disease. The animals may be humans as well as mammals such as cows, horses, sheep, pigs, goats, camels, antelopes, dogs, or cats, which are in need of treatment of symptoms similar to those of humans, without being limited thereto.

In the present invention, the “subject” may have senescent cells. In the present invention, the “subject” may have therapy-induced senescent cells.

In the present invention, the “subject” may have senescent cells induced by tumor cell senescence-inducing therapy. In the present invention, the subject might have been subjected to anticancer therapy, and may have senescent cells induced by the anticancer therapy.

In the present invention, the term “administration” means introducing the composition of the present invention to a subject by any suitable method. The pharmaceutical composition of the present invention may be administered orally or parenterally through various routes as long as it can reach a target tissue. The method of administration of the pharmaceutical composition according to the present invention is not particularly limited, but may follow the conventional methods used in the art. Regarding the method of administration, the pharmaceutical composition may be administered in a conventional manner, for example, via intravenous, intraarterial, intraperitoneal, intramuscular, intraarterial, intraperitoneal, intrasternal, transdermal, intranasal, inhalation, topical, intrarectal, oral, intraocular or intradermal routes, without being limited to the above examples.

In the present invention, the sample isolated from the subject may include tissue, cells, whole blood, serum, plasma or saliva isolated from the subject, and preferably may include tumor tissue or tumor spheroids, without being limited thereto.

### EXAMPLES

Hereinafter, the present invention will be described in more detail by way of examples. These examples are only intended to illustrate the present invention, and it will be apparent to those skilled in the art that the scope of the present invention is not to be construed as being limited by these examples.

### Example 1: Generation of Senescent Tumor Cells

To generate therapy-induced senescent tumor cells, breast cancer cell lines with contrasting characteristics (MCF7 and ZR75-1, which are female hormone receptor-positive but relatively less malignant, and Hs578T and MDA-MB-231, which are hormone receptor- and HER2 receptor-negative triple-negative breast cancer cell lines and are relatively more aggressive) were each treated with 100 nM of adriamycin (Selleckhem, Catalog No. S1208, CAS 25316-40-9). After 48 hours, the cell culture medium was replaced and the cells were cultured for 5 to 7 days, thereby generating therapy-induced senescent tumor cells.

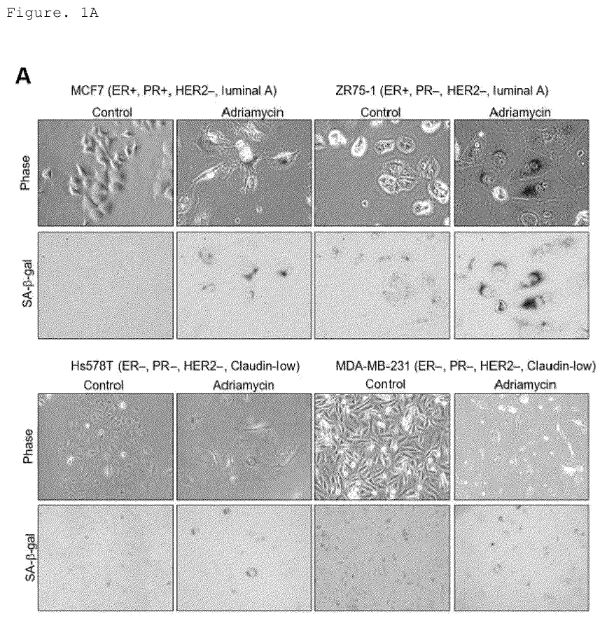

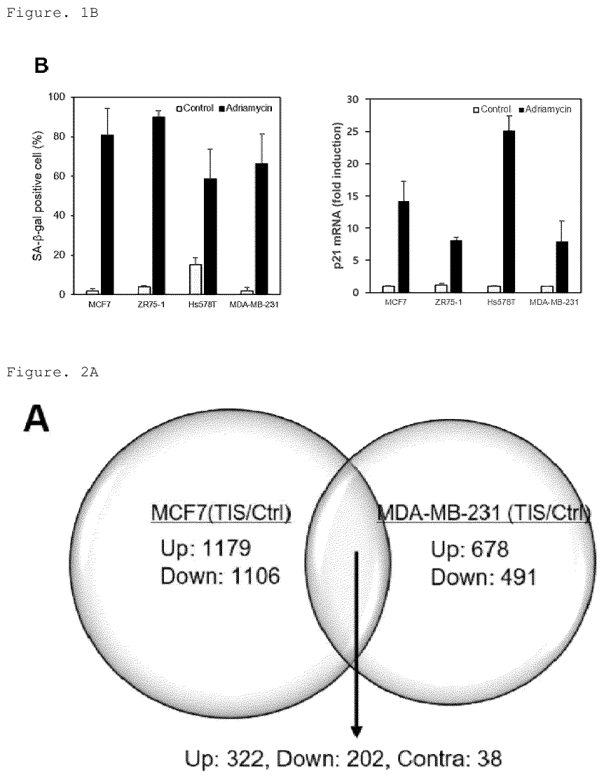

As shown in FIG. 1, it was confirmed that, after therapy-induced senescence by adriamycin, the cell size of each breast cancer cell line increased, and the number of cells positive to SA-b-gal, a senescence marker, increased. In addition, the mRNA expression of p21, a cell cycle inhibitor, was confirmed to increase significantly in each cell line, thereby obtaining therapy-induced senescent breast cancer cells.

### Example 2: Analysis of Gene Expression of Therapy-Induced Senescent Breast Cancer Cells and Selection of Drug Target

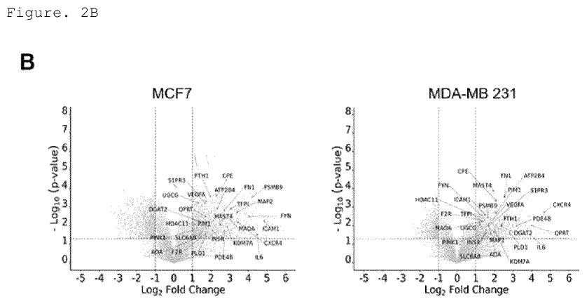

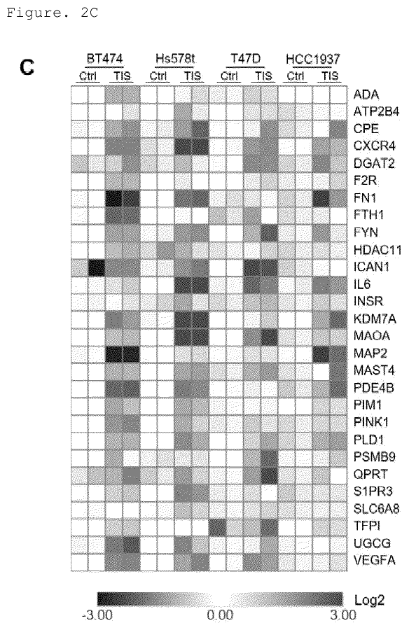

Next, gene expression of the therapy-induced senescent breast cancer cells generated in Example 1 was analyzed using RNASeq, and then 322 genes whose expression was increased in both cell lines were identified (FIG. 2A).

Among the 322 genes with increased expression, 28 genes targetable by FDA-approved drugs were selected, and when CF7 and MDA-MB 231 cell lines was induced to senescence by treatment, changes in the expression levels of the 28 genes were analyzed (FIG. 2B).

In addition to the MCF7 and MDA-MB 231 cell lines, other breast cancer cell lines, such as BT474, Hs578t, T47D, and HCC1937, were induced to senescence by treatment in the same manner as in Example 1, and changes in expression of the 28 genes were analyzed. The following 23 genes showing the same increasing expression pattern in at least two cell lines were finally selected as targets:

ATP2B4, CPE, CXCR4, DGAT2, F2R, FYN, ICAM1, IL6, INSR, KDM7A, MAOA, MAP2, MAST4, PDE4B, PIM1, PINK1, PLD1, PSMB9, QPRT, S1PR3, SLC6A8, UGCG and VEGFA.

### Example 3: Evaluation of Senolytic Effect of Drug for Each Target on Senescent Cells

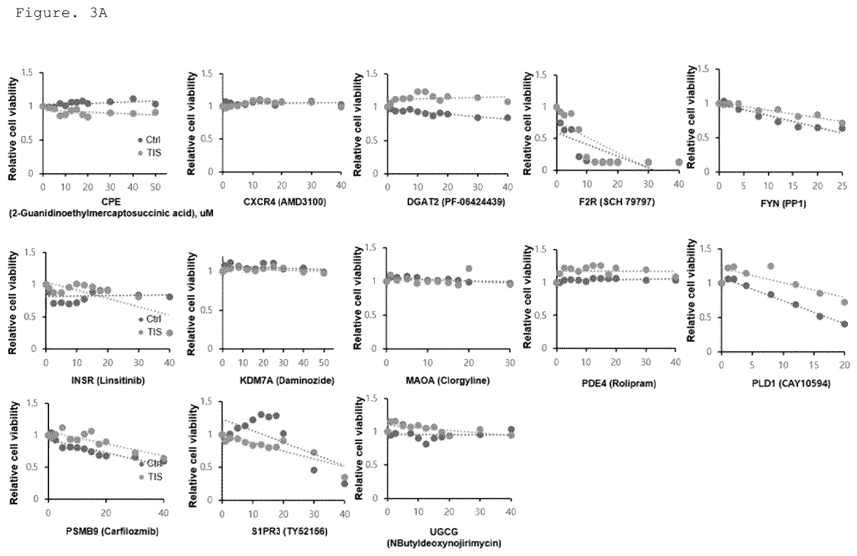

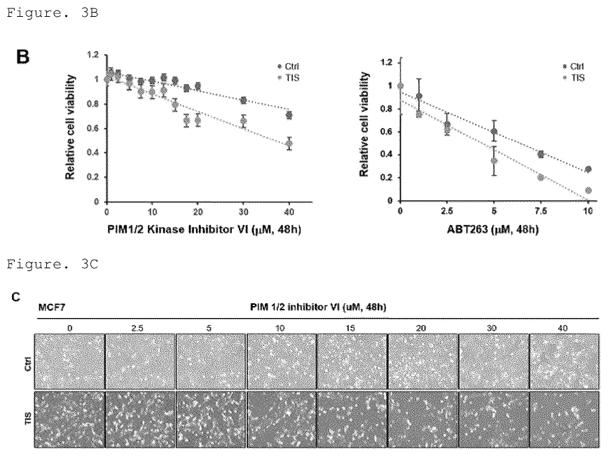

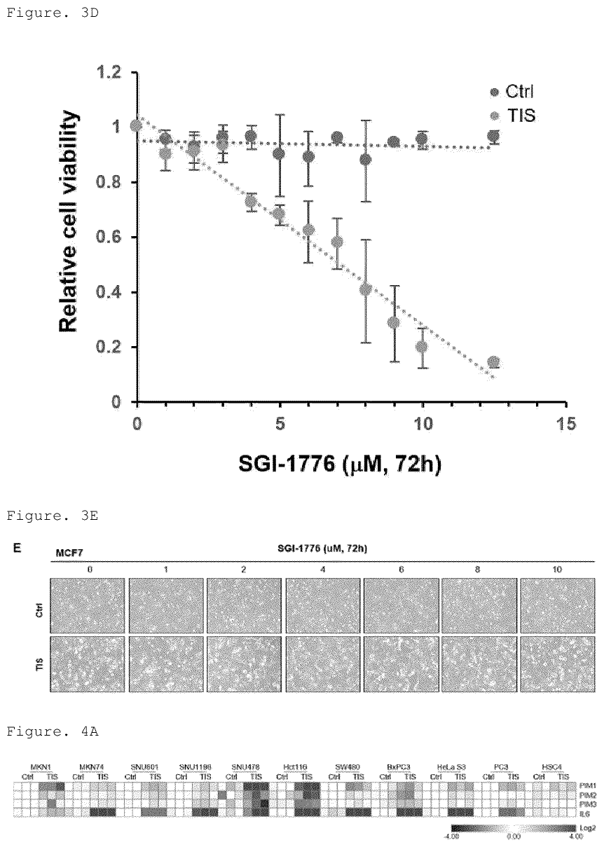

Therapy-induced senescent breast cancer cells were treated with the FDA-approved inhibitors (Table 1) for the 23 genes finally selected in Example 2, the senescent cell-selective cell division inhibitory effect and senolytic effect of the drugs were evaluated by CCK8 assay (D-Plus CCK, cell viability assay kit, Cat CCK-3000, Dong-In LS) (FIG. 3A). In this case, for the control group (cancer cells without therapy-induced senescence), the measurement was made after treatment with the inhibitor at the indicated concentration for 48 hours, and for the therapy-induced senescent cell group, the cancer cells were induced to senescence by treatment with adriamycin for 48 hours as described in Example 1, and then treated with the indicator at the indicated concentration for 48 hours while replacing the culture medium, the inhibitor was treated at the indicated concentration for 48 hours, and then the measurement was made.

TABLE 1

| Target | Inhibitors (NA: not available) |
|---|---|
| ATP2B4 | NA |
| CPE | 2-Guanidinoethylmercaptosuccinic acid |
| CXCR4 | AMD3100 |
| DGAT2 | PF-06424439 |
| F2R | SCH 79797 |
| FYN | PP1 |
| ICAM1 | NA |
| IL6 | Siltuximab |
| INSR | Linsitinib |
| KDM7A | Daminozide |
| MAOA | Clorgyline (hydrochloride) |
| MAP2 | NA |
| MAST4 | NA |
| PDE4B | Rolipram (ZK-62711) |
| PIM1 | PIM1/2 kinase inhibitor VI |
| PINK1 | NA |
| PLD1 | CAY10594 |
| PSMB9 | Carfilzomib |
| QPRT | NA |
| S1PR3 | TY 52156 |
| SLC6A8 | NA |
| UGCG | N-Butyldeoxynojirimycin•HCl |
| VEGFA | Bevacizumab |

As shown in FIG. 3A, as a result of analyzing cell viability after treating the control cell and the therapy-induced senescent tumor cells with the FDA-approved drugs known to inhibit the targets shown in Table 1, it was confirmed that only treatment with the PIM-1 kinase inhibitor exhibited a selective senolytic effect on the therapy-induced senescent tumor cells.

PIm-1 kinase was selected as the final target, and the selective senolytic effect on senescent cells was compared with that of ABT-263, a senolytic drug reported to be used in conventional “one-two punch” therapy. As a result, as shown in FIGS. 3B and 3C, it was confirmed that ABT 263 had a significant difference between the killing effect on the control cell and the killing effect on the TIS cells, but showed an almost complete killing effect on both the control and TIS cells, whereas the PIM-1 kinase inhibitor inhibited cell growth in the control cells at high concentrations but hardly killed the control cells, and effectively killed the TIS cells, indicating that the senolytic effect of the PIM-1 kinase inhibitor on the senescent cells was significantly superior to that of ABT-263.

In addition, in order to examine whether the use of PIM-1 kinase inhibitors other than PIM1/2 kinase inhibitor VI also exhibit the same senolytic effect as confirmed above, the selective senolytic effect of SGI-1776 (Selleckhem, Catalog No. S2198, CAS 1025065-69-3) on senescent cells was evaluated in the same manner. As shown in FIG. 3D, it was confirmed that even treatment with SGI-1776 (CAS 1025065-69-3) exhibited high cytotoxicity to the TIS cells compared to the control cells, indicating that PIM-1 kinase inhibitors can exhibit an excellent senolytic effect on senescent cells regardless of the types thereof.

### Example 4: Induction of Senescence of Various Cancer Cell Lines by Treatment, Analysis of Expression of PIM 1 Kinase, and Evaluation of Senolytic Effect

Unlike PIM kinases, secondary changes that regulate activity, such as phosphorylation, are not well known, and it has been reported that the overall activity is proportional to the expression level of PIM kinases.

In addition to breast cancer cell lines (MCF7 and MDA-MB231), gastric cancer (SNU601, MKN1, and MKN74), colon cancer (SW480, and Hct116), head and neck cancer (HSC4), cervical cancer (HeLa S3), cholangiocarcinoma (SNU308, SNU478, and SNU1196), pancreatic adenocarcinoma (BxPC3), and prostate cancer (PC3) cell lines were induced to senescence by treatment in the same manner as in Example 1, thereby obtaining therapy-induced senescent cells.

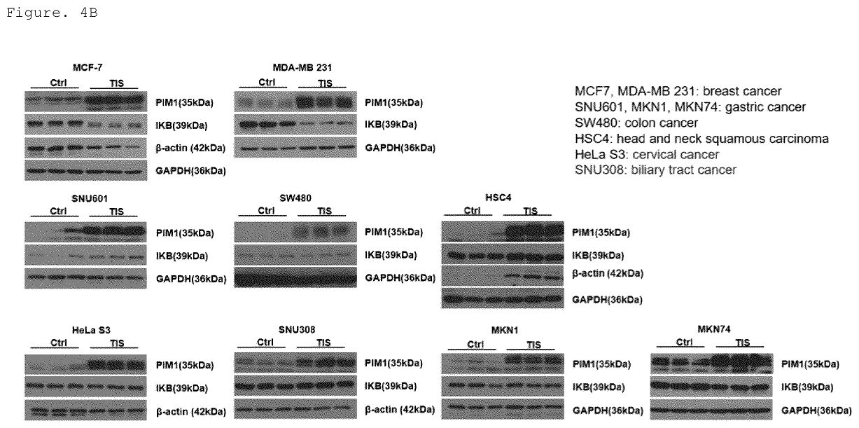

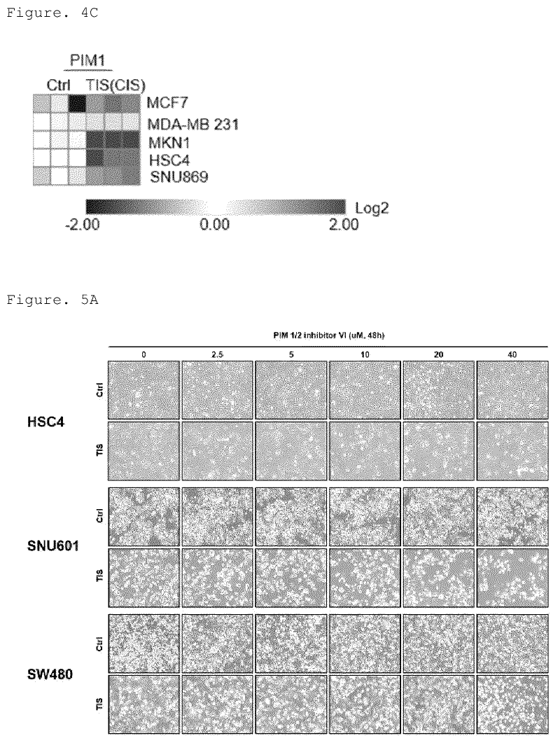

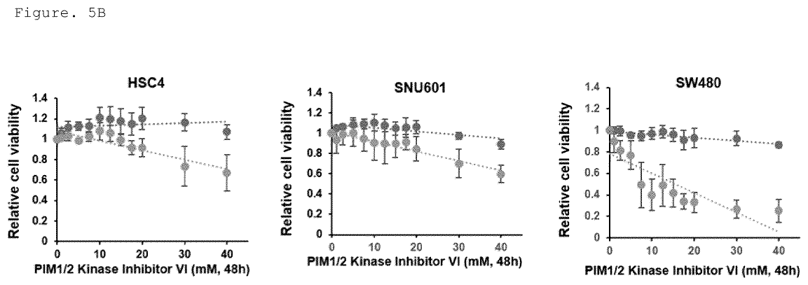

As a result, as shown in FIGS. 4a and 4b, it was confirmed that, when all of the cancer cell lines were induced to senescence by treatment, the mRNA and protein expression of PIM-1 kinase increased.

In addition, in order to examine whether therapy-induced senescence (TIS) occurs even when using anticancer agents other than adriamycin, various cancer cell lines (MCF7, MDA-MB 231, MKN1, HSC4, and SNU869) were treated with 10 μM of cisplatin (Selleckhem, Catalog No. S1166 CAS 15663-27-1). As a result, it was confirmed that therapy-induced senescence (TIS) occurred and mRNA expression of the PIM-1 kinase gene was significantly increased (FIG. 4c).

In order to evaluate the senolytic effect of a Pim-1 kinase inhibitor on gastric cancer (SNU601), head and neck cancer (HSC4), and colon cancer (SW480) cell lines induced to senescence by treatment, the concentration-dependent senolytic effect of the Pim-1 kinase inhibitor was evaluated in the same manner as described in Example 3.

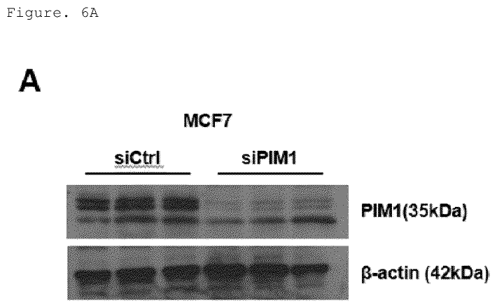

As shown in FIG. 5, it was confirmed that, in all of the cancer cell lines, the Pim-1 kinase inhibitor showed no significant killing effect on the control cells, whereas it exhibit a significant killing effect on the therapy-induced senescent cancer cells, indicating that the Pim-1 kinase inhibitor is able to selectively kill senescent cells.

### Example 4: Analysis of Selective Killing of Senescent Cells by Inhibition of Expression of Pim-1 Gene

In order to examine whether senescent cells can be selectively killed even by inhibition of expression of the Pim-1 gene other than treatment with the Pim-1 kinase protein inhibitor, expression of the Pim-1 gene was inhibited by treatment with siRNA (GUAUGAUAUGGUGUGUGGAGAUAUUC, SEQ ID NO: 1) targeting the Pim-1 gene, and cytotoxicity was evaluated.

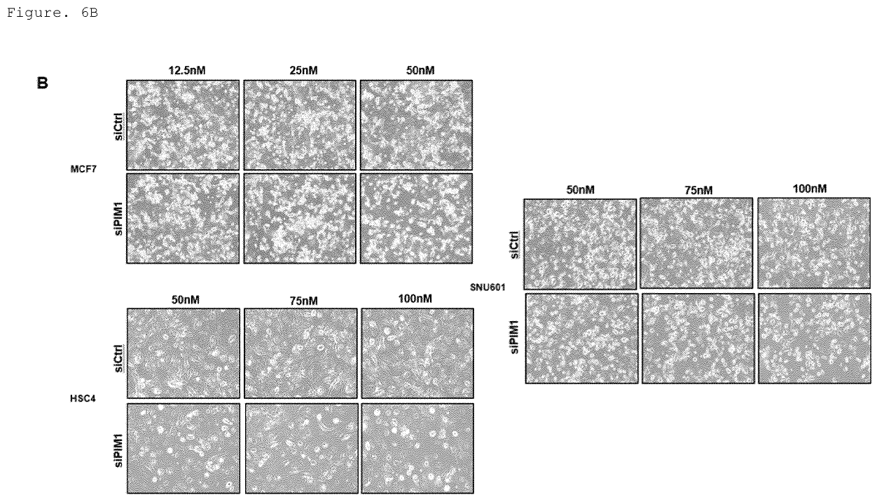

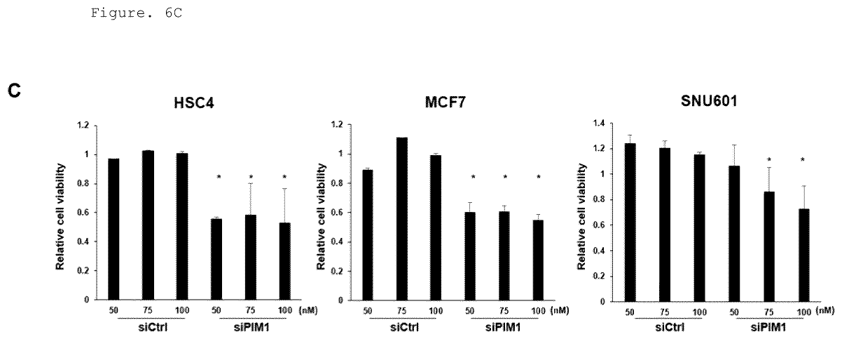

As a result, as shown in FIG. 6, it was confirmed that the expression of Pim-1 kinase could be inhibited (knocked down) at the gene level by treatment with the siRNA targeting the Pim-1 gene (FIG. 6a), and that, even when the expression of Pim-1 kinase was inhibited (knocked down) at the gene level by treating various cancer cells (MCF7, HSC4, and SNU601) with the siRNA targeting the Pim-1 gene, the siRNA exhibited a selective senolytic effect on the senescent cells (FIGS. 6b and 6c).

Considering that treatment with various compounds that inhibit the activity of Pim-1 kinase protein or with the siRNA that selectively inhibits the Pim-1 kinase gene consistently exhibited excellent growth inhibitory and killing effects on the senescent cells as described in the Examples of the present invention, it has been proven that inhibition of the expression or activity of Pim-1 kinase is a novel mechanism of action that can induce growth inhibition and apoptosis of senescent cells.

Therefore, from the results of the above Examples, it can be clearly understood that the Pim-1 kinase gene expression inhibitor or the Pim-1 kinase protein activity inhibitor, regardless of the type and form thereof, may be used to kill various senescent cells (senolytic use) through inhibition of Pim-1 PP-B2966.docx kinase (Ajou University).

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### INDUSTRIAL APPLICABILITY

The composition for killing senescent cells comprising a Pim-1 kinase inhibitor according to the present invention exhibits a selective killing effect on senescent cells, particularly senescent cells with increased expression of Pim-1 kinase. The composition for killing senescent cells according to the present invention may be effectively used to prevent or treat senescence-related diseases through its selective killing effect on senescent cells. In particular, the composition may exhibit an excellent effect on the prevention or treatment of tumors by selectively killing therapy-induced senescent cells by administering the Pim-1 kinase inhibitor subsequent to anticancer therapy based on the induction of senescence of tumor cells.

### Sequence Listing Free Text

Electronic file attached.

1. A method for killing senescent cells in a subject in need thereof, comprising administering a Pim-1 kinase inhibitor to the subject.

2. The method according to claim 1, wherein the Pim-1 kinase inhibitor is a Pim-1 kinase gene expression inhibitor or a Pim-1 kinase protein activity inhibitor.

3. The method according to claim 2, wherein the Pim-1 kinase gene expression inhibitor is selected from the group consisting of antisense nucleotides, siRNAs (small interfering RNAs), shRNAs (small hairpin RNAs), ribozymes, aptamers, anti-microRNAs, microRNA mimics, nucleases, zinc finger nucleases (ZFNs), transcription activator-like effector nucleases (TALENs), gRNA, sgRNA, and self-ribozyme-flanked RNAs, which target PIM-1 kinase gene.

4. The method according to claim 3, wherein the Pim-1 kinase gene expression inhibitor is an siRNA comprising a sequence set forth in SEQ ID NO: 1.

5. The method according to claim 2, wherein the Pim-1 kinase protein activity inhibitor is selected from the group consisting of compounds, peptides, peptidomimetics, substrate analogs, aptamers, and antibodies and fragments thereof, which specifically inhibit the activity of the Pim-1 kinase protein.

6. The method according to claim 1, wherein the senescent cells are senescent cancer cells.

7. The method according to claim 6, wherein the senescent cancer cells are oncogene-induced senescent cells (OIS cells) or therapy-induced senescent cells (TIS cells).

8. The method according to claim 1, wherein the senescent cells have increased expression of Pim-1 kinase.

9. The method according to claim 7, wherein the therapy-induced senescent cells (TIS cells) are cells induced to senescence by chemotherapy, biological therapy, or radiotherapy.

10. The method according to claim 7, wherein the therapy-induced senescent cells (TIS cells) are cancer cells induced to senescence by an anticancer agent.

11. A method for preventing or treating a senescence-related disease in a subject in need thereof, comprising administering a Pim-1 kinase inhibitor to the subject.

12. The method according to claim 11, wherein the senescence-related disease is selected from the group consisting of Alzheimer's disease, Parkinson's disease, cataracts, macular degeneration, glaucoma, arteriosclerosis, acute coronary syndrome, myocardial infarction, stroke, hypertension, idiopathic pulmonary fibrosis (IPF), chronic obstructive pulmonary disease (COPD), osteoarthritis, type 1 or type 2 diabetes, obesity, adipose dysfunction, coronary artery disease, cerebrovascular disease, periodontal disease, atrophy in various tissue, cancer therapy-related disorders such as fibrosis, brain and heart damage, and myelodysplastic syndrome.

13. A method for preventing or treating a tumor in a subject in need thereof, comprising administering a Pim-1 kinase inhibitor to the subject, after tumor cell senescence-inducing therapy.

14. The method to claim 13, wherein the Pim-1 kinase inhibitor is a Pim-1 kinase gene expression inhibitor or a Pim-1 kinase protein activity inhibitor.

15. The method according to claim 14, wherein the Pim-1 kinase gene expression inhibitor is selected from the group consisting of antisense nucleotides, siRNAs (small interfering RNAs), shRNAs (small hairpin RNAs), ribozymes, aptamers, anti-microRNAs, microRNA mimics, nucleases, zinc finger nucleases (ZFNs), transcription activator-like effector nucleases (TALENs), gRNA, sgRNA, and self-ribozyme-flanked RNAs, which target PIM-1 kinase gene.

16. The method according to claim 14, wherein the Pim-1 kinase gene expression inhibitor is a siRNA comprising a sequence set forth in SEQ ID NO: 1.

17. The method according to claim 14, wherein the Pim-1 kinase protein activity inhibitor is selected from the group consisting of compounds, peptides, peptidomimetics, substrate analogs, aptamers, and antibodies and fragments thereof, which specifically inhibit the activity of the Pim-1 kinase protein.

18. The method according to claim 13, wherein the tumor cell senescence-inducing therapy is chemotherapy, biological therapy, or radiotherapy.

19. The method according to claim 13, wherein the Pim-1 kinase inhibitor selectively kills therapy-induced senescent tumor cells (TIS tumor cells) generated by the tumor cell senescence-inducing therapy.

20. The method according to claim 13, wherein the tumor cell senescence-inducing therapy induce increasing an expression of Pim-1 kinase in tumor cells.


SEQUENCE LISTING (1 sequence)

SEQ ID NO: 1 - RNA, 26 nt, synthetic construct
  source 1..26: mol_type = other RNA
gtatgatatggtgtgtggagatattc